(19) United States
(12) Patent Application Publication
(10) Pub. No.: US 2010/0174191 A1
(43) Pub. Date: Jul. 8, 2010

(54) APPARATUS AND METHOD FOR PROVIDING A DYNAMIC 3D ULTRASOUND IMAGE

(75) Inventors: Chin-An LIN, Taipei City (TW); Guo-Zua Wu, Taichung City (TW); Sun-Hua Pao, Taipei City (TW); Chien-Hsing Huang, Zhudong Township (TW); Hsing-Cheng Yu, Hukou Township (TW); Hung-Yueh Chen, Fengyuan City (TW)

Correspondence Address: MICHAEL W. TAYLOR, P.O. BOX 3791, ORLANDO, FL, 32802-3791, US

(73) Assignee: Industrial Technology Research Institute, Chutung (TW)

(21) Appl. No.: 12/632,156

(22) Filed: Dec. 7, 2009

(30) Foreign Application Priority Data
Dec. 16, 2008 (TW) 097149064

(51) Int. Cl. A61B 8/14; A61B 5/0402

(52) U.S. Cl. 600/443; 600/509

(57) ABSTRACT

A system and method thereof for capturing and reconstructing a dynamic 3D ultrasound image of a blood vessel are provided. The present system includes a ultrasound transducer, a motor positioning system, an electrocardiograph, a microprocessor for processing ultrasound images and signals capturing by the ultrasound transducer, and a monitor displaying the dynamic 3D ultrasound image. The motor positioning system controls the ultrasound transducer to capture 2D ultrasound images of the blood vessel at several locations in a predetermined urea. During the capturing process, capturing times, electrocardiograms and 3D locations of those captured 2D ultrasound images are simultaneously recoded. Finally, those captured 2D ultrasound images are reconstructed to the dynamic 3D ultrasound image of the blood vessel according to the time bases based on the phases of the electrocardiograms.

### TECHNICAL FIELD

The present disclosure relates to an apparatus for capturing and reconstructing a three-dimensional (3D) ultrasound image of a vessel and the method thereof. More particularly, the present apparatus and method are used for imaging and displaying a dynamic 3D ultrasound image of the vessel.

### DESCRIPTION OF RELATED ART

The ultrasonic detection is popular in medical procedure since its characteristics of cheapness and noninvasion. Presently, the ultrasonic detection is performed via repeatedly scanning a specific region of the patient with the handheld ultrasonic transducer. During the ultrasonic detection, the piezoelectric transducer of the ultrasonic transducer generates ultrasonic waves with 2 to 13 MHz to introduce into the patient's body. Then, the oscillation of the ultrasonic transducer receives the ultrasonic echoes reflexed from the interfaces of various tissues and transforms thereto electronic pulses. The electronic pulses are sequentially transmitted to the processor and operated to be the digital images.

Two-dimensional (2D) ultrasonic images are widely applied in ultrasonic detections. However, if 2D ultrasonic images are used to be the bases for further diagnoses, the information obtained therefrom seem insufficient therefor since the power of ultrasonic waves will apperently lose while those ultrasonic waves meet the calcified occlusion. Thus, the near total occlusion of artery and the complete occlusion of artery are hardly differentiated therefrom simply based on 2D ultrasonic images.

The acquisitions of ultrasonic images are usually triggered by a series of pulsing signals in the ultrasonic detection of vessel. These pulsing signals can be generated according to the electrocardiogram, the heartbeat, the time counting clock or heart-related data. For example, the electrocardiogram (ECG) can be defined as specific waves and/or phases which are corresponding to the systole and the diastole. The R-wave and P-wave in ECG respectively express the start of systole and the end of diastole. Accordingly, the data of ECG is appropriate to be the references to ascertain the phases of systole and diastole.

The related art of U.S. Pat. No. 7,302,286 is shown in FIG. 1. When demonstrates a dynamic image of transformable organ, in this invention, the cross-sectional images of the dynamic image must be acquired at an identical one of phases of the cardiac cycle. For example, the way of tracing the R-wave in ECG is applied in this invention. The dynamic image of this invention can be used for measuring the volume of the transformable organ on the same conditions.

As shown in FIG. 2, Patent of U.S. Pat. No. 6,692,438 provides the related art that the electrocardiograph triggers the ultrasonic equipment to acquire the ultrasonic images at predetermined phases of cardiac cycle and records the phases of cardiac cycle simultaneously. By the invention, a series of ultrasonic images of the organ at known phases of cardiac cycle are obtained via acquiring ultrasonic images at the target region for a duration of several cardiac cycles.

The related art of U.S. Pat. No. 7,415,093 is shown in FIG. 3. In this related art, the cardiac cycles are observed and recorded by the electrocardiograph and the endocardiography prior to perform the computed tomography (CT). The principles of CT are utilizing the respective degrees of absorption of X-ray of various tissues to generate cross-sectional image, which can be the materials of medical diagnosis. In U.S. Pat. No. 7,415,093; the acquired electrocardiogram and ultrasonic images of heart are analyzed simultaneously for the synchronization of the phases of cardiac cycle and triggering acquisitions of images of CT when the CT process is performed.

Employing experiments and researches full-heartily and persistently, the applicant finally conceived preferable apparatus and method for providing a dynamic 3D ultrasound image.

### SUMMARY OF THE INVENTION

The present disclosure provides a method of imaging a three-dimensional (3D) ultrasonic image of a vessel comprising the steps of providing a cardiac cycle time of a subject; providing a plurality of ultrasonic images acquired at a plurality of successive positions of the vessel, wherein the plurality of ultrasonic images corresponding to the plurality of positions are acquired during a plurality of acquiring time periods each of which is at least equal to the cardiac cycle time, and providing a positioning system to position each of the plurality of positions; providing a plurality of sets of cardiac cycles respectively corresponding to the acquiring time periods, wherein each of the plurality of cardiac cycles has a plurality of unit phases; catching a specific ultrasonic image for each of the plurality of positions, wherein the specific ultrasonic image corresponds to a specific one of the plurality of unit phases; and reconstructing the specific ultrasonic images for the plurality of positions to provide the 3D ultrasonic image of the vessel.

On another aspect, the present disclosure provides a method of imaging a three-dimensional (3D) ultrasonic image comprising the steps of positioning a plurality of positions along a target region; providing a plurality of ultrasonic images captured at the plurality of positions; catching a specific ultrasonic image for each of the plurality of positions; and reconstructing the specific ultrasonic images of the plurality of positions to provide the 3D ultrasonic image.

On another aspect, the present disclosure provides system imaging a three-dimensional (3D) ultrasonic image of a target region comprising an ultrasonic transducer providing a plurality of ultrasonic images classified into a plurality of image sets each of which has specific ultrasonic images revealing a specific position of the targeting region; a positioning system positioning the specific positions; and a processor generating the 3D ultrasonic image by reconstructing from a respective first one of the specific ultrasonic images of each of the plurality of image sets.

### BRIEF DESCRIPTION OF THE DRAWINGS

FIG. 1 is a diagram showing the related art of Patent of U.S. Pat. No. 7,302,286;

FIG. 2 is a diagram showing the related art of Patent of U.S. Pat. No. 6,692,438;

FIG. 3 is a diagram showing the related art of Patent of U.S. Pat. No. 7,415,093;

FIG. 4(A) is a flow chart illustrating the method of the present disclosure for acquiring the ultrasonic images and

FIG. 4(B) shows a series of ultrasonic images 161 at a specific position;

FIG. 5 is a flow chart illustrating the method of the present disclosure for reconstructing a 3D ultrasonic image from a series of 2D ultrasonic images;

6(A) to (C) are the diagrams showing the results of the process of the present method of FIG. 5;

FIG. 7 is a diagram showing a device imaging a three-dimensional (3D) ultrasonic image of the present disclosure; and

FIG. 8 is a diagram demonstrating the demarcation of the peripheral arterioles of human kidney.

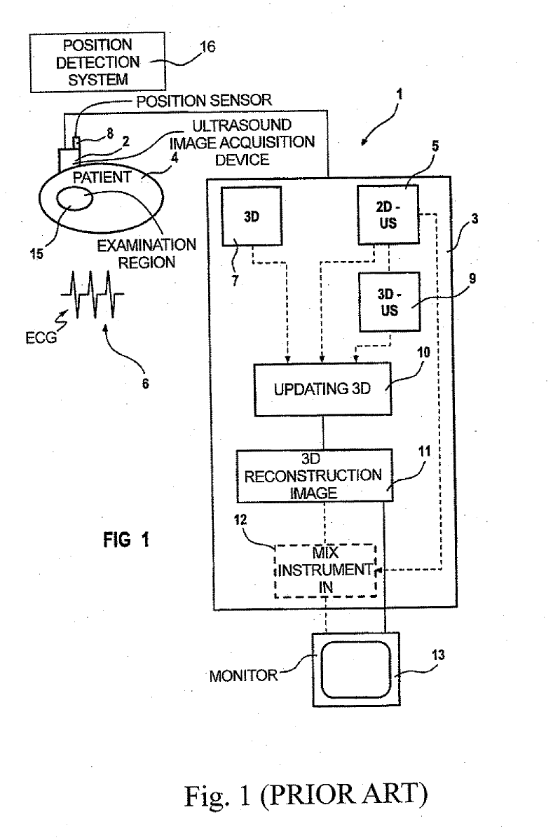

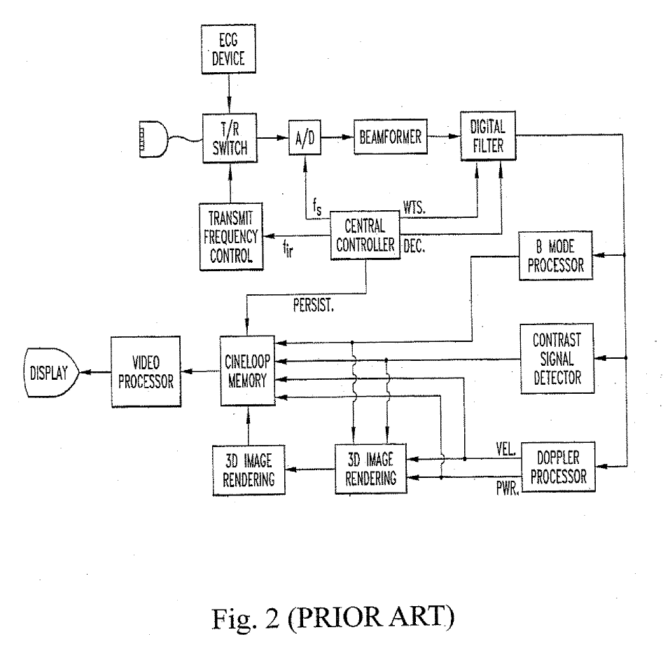

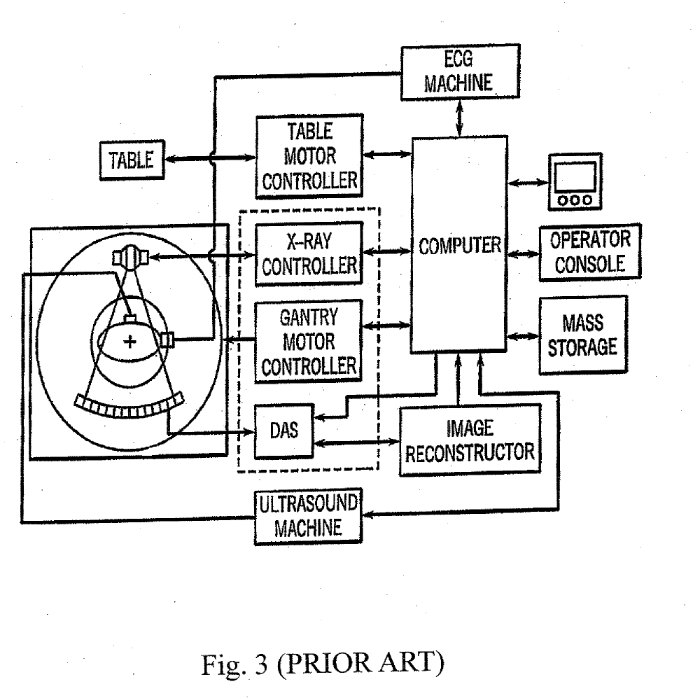

### DESCRIPTION OF THE EMBODIMENTS

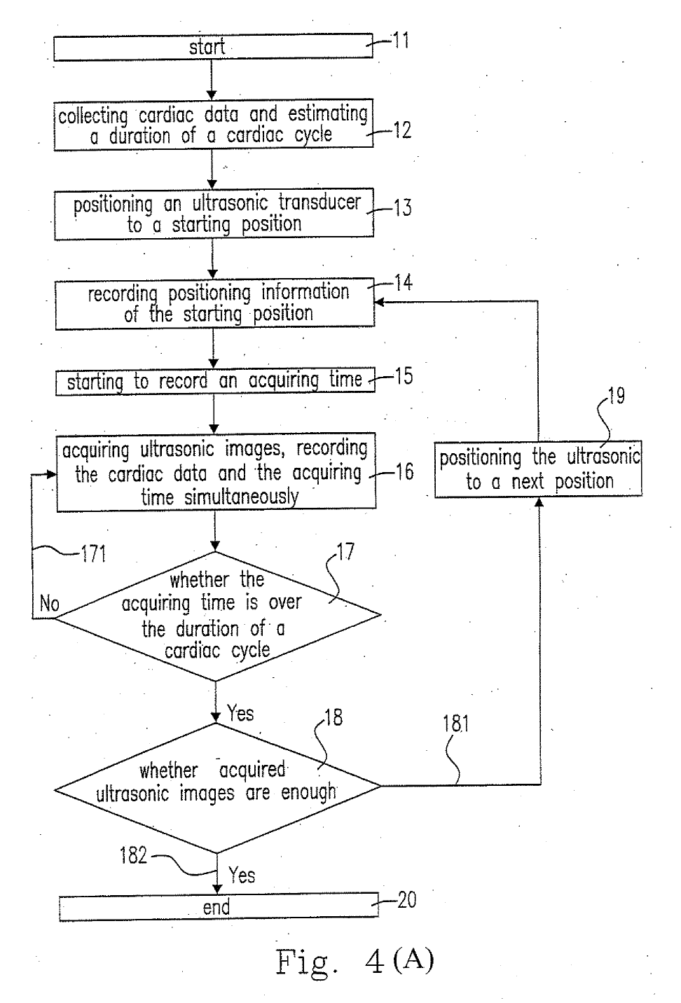

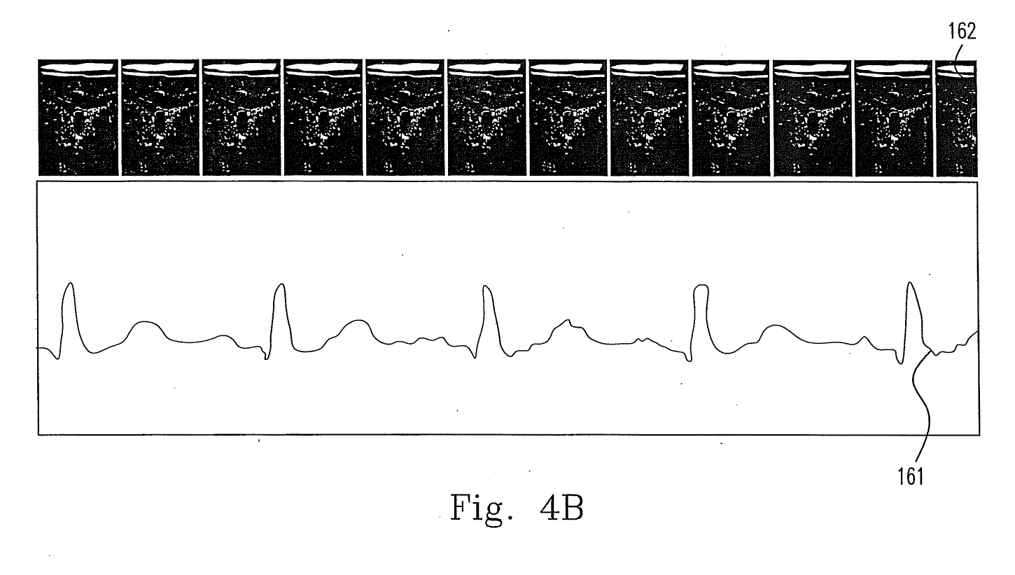

Please refer to FIG. 4(A), which is a flow chart illustrating the method of the present disclosure for acquiring the ultrasonic images. When start to acquire ultrasonic images (Step 11), a duration of one cardiac cycle of a subject is obtained by the subject's cardiac data, e.g. an electrocardiogram (Step 12). Then, an ultrasonic transducer will be posited to a starting position of target region on the shin (Step 13), where the positing process can be driven by an auxiliary system such as a positioning system having an electronic motor and positioning information of the starting position is recorded (Step 14), and the target region is typically a vessel or an organ. After the ultrasonic transducer is well posited, an acquiring time, i.e. the time for acquiring ultrasonic image, starts to be recorded (Step 14), meanwhile, the ultrasonic transducer is triggered to acquire ultrasonic image (Step 15). By the ultrasonic transducer, the ultrasonic images at the start position will continuously be acquired for about one second which is approximately over the normal duration of one cardiac cycle; meanwhile, the cardiac data and the acquiring time will be recorded simultaneously (Step 16).

FIG. 4(B) shows a series of ultrasonic images 161 at a specific position, e.g. the start position, of a vessel and the electrocardiogram 162 simultaneously recorded during the duration of the series of ultrasonic images 161 being acquired, which also illustrates the ultrasonic images and the cardiac data acquired in Step 16. As shown in FIG. 4(B), electrocardiogram 162 includes several cardiac cycles and the series of ultrasonic images 161 are continuously acquired at the specific position of the vessel during the duration of the several cardiac cycles. In other words, after the ultrasonic transducer acquires respective ultrasonic images at various positions of the vessel, i.e. processing Step 16 for several times, a plurality of image sets respectively including specific ultrasonic images of one of the various positions of the vessel will be obtained.

Please still refer to FIG. 4 (A). In Step 17, the acquiring time for the ultrasonic transducer acquiring ultrasonic images at the start position is checked whether it is over the duration of one cardiac cycle. If the result of Step 17 is “No” 171, the ultrasonic transducer will continuously acquires ultrasonic images at the start position. If the result of Step 17 is “Yes” 172, then the ultrasonic images acquired in Step 16 are analyzed for determining whether those acquired ultrasonic images are enough for revealing the target region. If the result of Step 18 is “No” 181, the ultrasonic transducer is moved to a next position of the target region (Step 19) for repeating Step 14 to Step 18 to obtain the specific ultrasonic images of the next position. By repeating Step 14 to Step 19, a series of specific ultrasonic images respectively revealing various positions of target region and the electrocardiograms corresponding to the acquiring times of the series of specific ultrasonic images are obtained.

If the result of Step 18 is “Yes” 182, it is shows that the acquired ultrasonic images are enough for following processes such as reconstructing the acquired ultrasonic images to image a three-dimensional (3D) ultrasonic image of the target region and the acquisition of ultrasonic image will be finished (Step 20).

In the repeat of Step 14 to Step 19, the various positions of target region are adjacent from one another, and the movements of the ultrasonic transducer around the various positions in Step 19 is driven by the positioning system so as to accurately position the ultrasonic transducer at the various positions, where the 3D positions of the ultrasonic transducer in Step 19 and the acquired ultrasonic images are also recorded. Those information of 3D positions will increase the accuracy of the applications of the acquired ultrasonic images like imaging the 3D ultrasonic image as mentioned above.

When imaging the 3D ultrasonic image, in the present disclosure, the electrocardiogram is applied to be the timeline. Accordingly, in Step 17, the ultrasonic transducer needs continuously acquiring ultrasonic images over the duration of one cardiac cycle at each of the various positions of the target region.

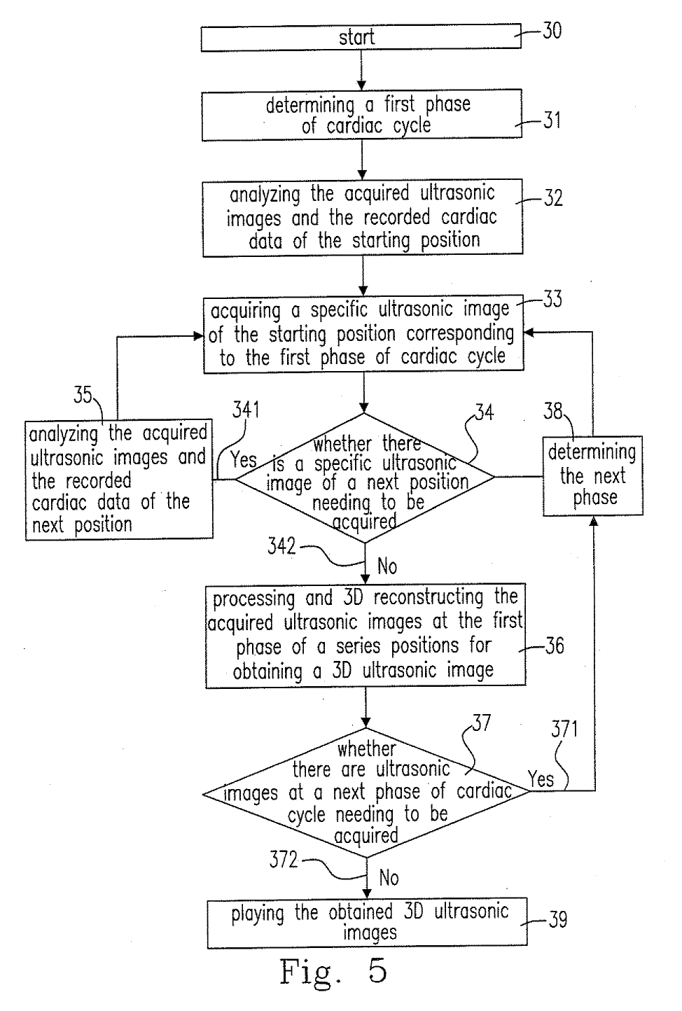

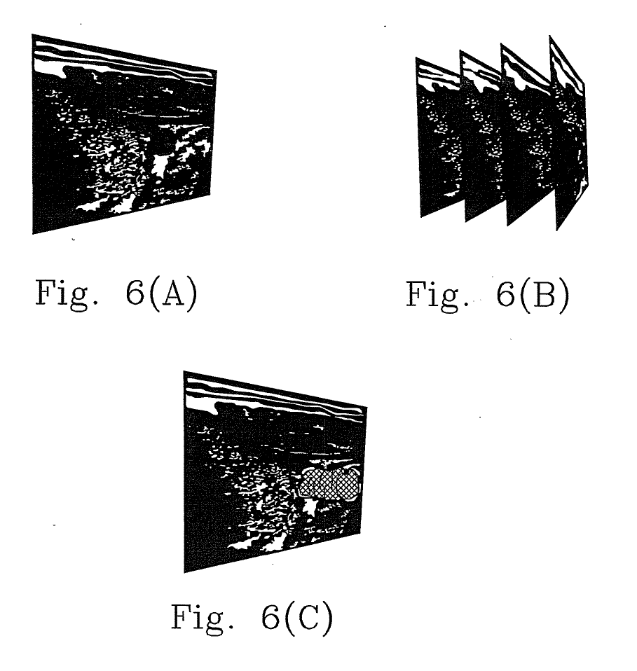

Please refer to FIG. 5, which is a flow chart illustrating the method of the present disclosure for reconstructing a 3D ultrasonic image from a series of 2D ultrasonic images. When starting the reconstructing process of 3D ultrasonic image. (Step 30), a first one of phases of the cardiac cycle is determined (Step 31). Next, the ultrasonic images acquired at the starting position and the electrocardiogram recorded simultaneously therewith are analyzed (Step 32). Then, via the recorded electrocardiogram being the timeline, a specific one of the ultrasonic images acquired at the starting position corresponding to the first phase of the cardiac cycle is caught (Step 33).

After the specific ultrasonic image of the starting position corresponding to the first phase is caught, it will be checked whether there is another specific ultrasonic image of a next position of the target region corresponding to the first phase needing to be acquired (Step 34). If the result of Step 34 is “Yes” 341, the ultrasonic images of the next position and the simultaneously recorded electrocardiogram are analyzed. (Step 35) and the specific ultrasonic image of the next position identically corresponding to the first phase is caught in Step 33. If the result of Step 34 is “No” 342, it is revealed that the respective specific ultrasonic images caught at all of the predetermined positions of the target region are completely collected. By the confirmation of “No” 342, the respective ultrasonic images acquired at Step 33 to Step 35, i.e. the ultrasonic images of each of the predetermined positions of the target region corresponding to the same one phase of the cardiac cycle, will be processed and three-dimensional (3D) reconstructed (Step 36). After 3D reconstructing the ultrasonic images of each of the predetermined positions corresponding to the same cardiac cycle phase, i.e. finishing the process of Step 30 to Step 36 once, an accurate 3D ultrasonic image of the target region, i.e. the vessel, at a specific time is obtained.

Please refer to FIG. 5. when Step 36 is finished, it will be confirmed whether there is a next phase of the cardiac cycle needing to be analyzed for another 3D image reconstruction (Step 37). If the result of Step 37 is “Yes” 371, the next phase of the cardiac cycle will be determined (Step 38). After the next cardiac cycle phase is determined, the process of the present method will perform Step 32 to Step 36 to acquire the necessary ultrasonic images for 3D image reconstruction of the next cardiac cycle phase until all of 3D ultrasonic images of the predetermined cardiac cycle phases are obtained. At this time moment, the result of Step 37 will be “No” 372.

As to the process of the present method of acquiring ultrasonic images at various positions and the same cardiac cycle phase, for example, P wave can be the specific one of the cardiac cycle phases. By acquiring and reconstructing all of the ultrasonic images at each of the predetermined positions of the vessel, the 3D ultrasonic image at the time of P wave can be obtained. Based on the same process, the 3D ultrasonic images of other cardiac cycle phases, e. g. R wave or T wave, will be easily obtained. In addition, since the respective acquiring times, i.e. the duration of acquiring ultrasonic images, of every predetermined positions of the target region are controlled to be approximately over the duration of one cardiac cycle so that the respective ultrasonic images corresponding to every predetermined cardiac cycle phases at the every predetermined positions can be acquired.

Please still refer to FIG. 5. When the respective 3D ultrasonic images corresponding to each of the predetermined cardiac cycle phases are obtained, those obtained respective 3D ultrasonic images will be played with the order of cardiac cycle phases so as to generate a dynamic 3D ultrasonic images pulsing with the cardiac cycle, i.e. the heartbeat.

Please refer to FIG. 6(A) to (C), which are the diagrams showing the results of the process of the present method of FIG. 5. FIG. 6(A) shows the result of Step 32 and Step 33 and is an acquired 2D ultrasonic image corresponding to a specific position and a specific cardiac cycle phase. FIG. 6(B) shows the respective 2D ultrasonic images corresponding to various positions but to the same cardiac cycle phase, which is the result obtained from Step 34 to “Yes” 341 to Step 35 to Step 33. FIG. 6(C) is the result of Step 36 showing the reconstructed 3D ultrasonic image where the vessel is separated and the surface of vessel is drawn, wherein the 3D ultrasonic image reveals the vessel at the time moment of the specific cardiac cycle phase.

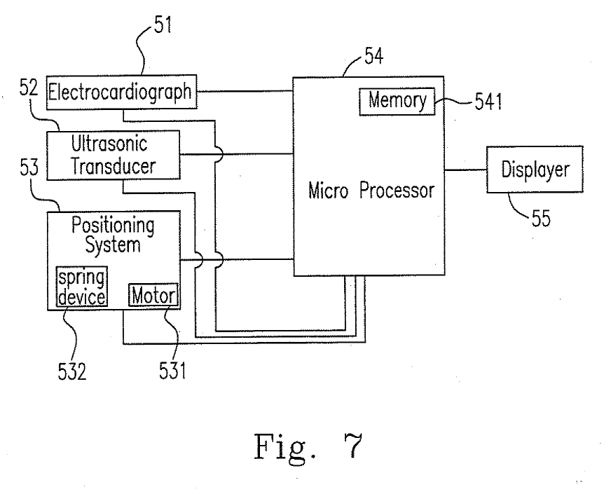

Please refer to FIG. 7, which is the diagram showing a device 50 imaging a three-dimensional (3D) ultrasonic image of a target region. Imaging device 50 includes an electrocardiograph 51, an ultrasonic transducer 52, a positioning system 53, a micro processor 54 and a displayer 55, wherein positioning system 53 further includes at least one of motor 531 and a spring element 532, micro processor 54 further includes a memory 541 and electrocardiograph 51, ultrasonic transducer 52, positioning system 53, micro processor 54 and displayer 55 are electrically connected. By performing the steps shown in FIGS. 4 and 5, imaging device 50 can generate a 3D ultrasonic image and/or a dynamic 3D ultrasonic image.

Ultrasonic transducer 52 is replaceable with an appropriate one depending on the location of the target vessel and performs various modes such as B mode, M mode, direct color mode, power color mode, direct power color mode and spectral Doppler mode. The frequency generated from ultrasonic transducer 52 is controlled form 20 KHz to 50 KHz. During the images acquiring process, ultrasonic transducer 52 acquires 20 to 30 ultrasonic images per second, which depends on the distance from the target vessel to the skin.

In the embodiment of the present disclosure, the image acquiring position of ultrasonic transducer 52 is controlled and positioned by positioning system 53. Positioning system 53 includes two motor one of which drives ultrasonic transducer 52 moving along the predetermined positions on the skin and another one drives ultrasonic transducer 52 rotating along an axis which is the connecting region of ultrasonic transducer 52 and the skin. The two mentioned motor of positioning system 53 are able to position the ultrasonic transducer 52. According to the positioning and rotating information of ultrasonic transducer 52, the 3D positions of every points on the acquired ultrasonic image are obtained. Moreover, ultrasonic transducer 52 can couple to a suspensory system for adjusting the height of ultrasonic transducer 52 so that ultrasonic transducer 52 will appropriately stay on the subject's skin.

The density of the vessel can also be estimated by the dynamic 3D ultrasonic image of the present disclosure. In the tumor, the blood flowing in the vessel can be transferred to be revealed as the images via the Doppler frequency, and the density of the vessel is quantified by those images. However, the vessel of the tumor is asynchronous with the perfusion so that the density of the vessel at a specific time moment will be underestimated. For example, even during the systole at which the heart pumps out the most blood and the blood in the tumorous vessel is at the maximum, the density of the vessel is sometimes underestimated for 30% compared with the real quantity. Therefore, it is necessary to acquire and record the distributions of the tumorous vessels at various times according to the timeline of the electrocardiogram. Prior to the analysis of 3D ultrasonic images, all of the images are offset adjusted on the position according to the similarities between the images. The offset adjustments on the position is performed by the convolution of the position and the lightness of pixel of continuous images to obtain a maximum correlation coefficient being the reference. Then, the distribution of tumorous vessels is obtained from the union of several cycles. In addition, for avoiding the erroneous adjustment on the interferences of image, the number of times of heartbeat is provided being the threshold. If the signals of the vessel happening at one region of a series of 3D ultrasonic images lower than the number of times of heartbeat, these signals should be ignored.

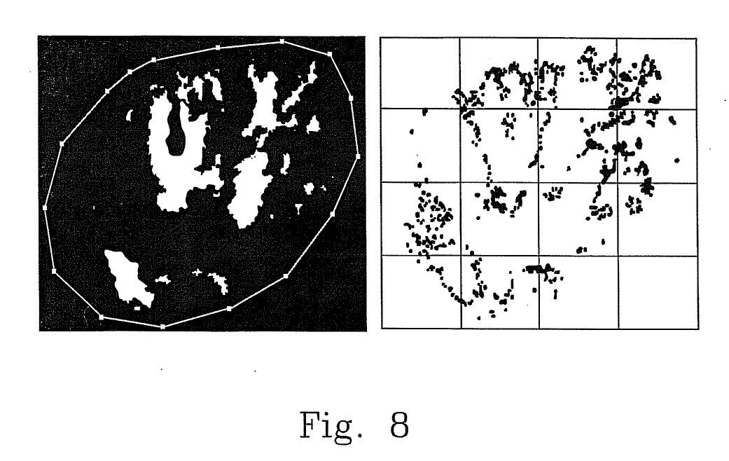

Moreover, the 3D ultrasonic images of the present disclosure can also be the materials for estimating the density of the arteriole. According to the clinical data, the density of the arteriole is helpful for distinguishing the difference between the benign and metastatic tumors. However, the arteriole is hard to be separated under the Doppler frequency. Moreover, the blood in the arteriole is pulsing-flowed so that the average flowing speed of the blood is difficultly estimated and defined. For filtering the interferences of low frequency, e.g. erroneous estimations of signals of blood generated by the breath or the pulse of vascular wall, the medical ultrasonic device is configured with the wall filter where the threshold of flowing speed thereof is set about 2 cm/sec which is also the threshold of the present disclosure for quantifying the density of the arteriole. The vessel is imaged due to the flowing speed of the blood being higher than the threshold. Accordingly, based on the matter that both the flowing speed and the volume of blood are relatively high at the systole, the volumes of arteriole respectively estimated at the systole and the diastole are respectively defined as V_{systole} and V_{diastole}. The difference of the volume of arteriole, V_{d}, is calculated by V_{systole}-V_{diastole}. By demonstrating on the human kidney, as shown in FIG. 8, the peripheral arterioles of human kidney are demarcated. Moreover, if V_{d} is applied to estimate the density of the arteriole, the difference between the benign and metastatic tumors are more distinctly revealed (n=65, P<0.004).

While the disclosure has been described in terms of what is presently considered to be the most practical and preferred embodiments, it is to be understood that the disclosure needs not be limited to the disclose embodiments. Therefore, it is intended to cover various modifications and similar arrangements included within the spirit and scope of the appended claims, which are to be accorded with the broadest interpretation so as to encompass all such modifications and similar structures.

What is claimed is:

1. A method of imaging a three-dimensional (3D) ultrasonic image of a vessel, comprising the steps of:
(a) providing a cardiac cycle time of a subject;
(b) providing a plurality of ultrasonic images acquired at a plurality of successive positions of the vessel, wherein the plurality of ultrasonic images corresponding to the plurality of positions are acquired during a plurality of acquiring time periods each of which is at least equal to the cardiac cycle time, and providing a positioning system to position each of the plurality of positions;
(c) providing a plurality of sets of cardiac cycles respectively corresponding to the acquiring time periods, wherein each of the plurality of cardiac cycles has a plurality of unit phases;
(d) catching a specific ultrasonic image for each of the plurality of positions, wherein the specific ultrasonic image corresponds to a specific one of the plurality of unit phases; and
(e) reconstructing the specific ultrasonic images for the plurality of positions to provide the 3D ultrasonic image of the vessel.

2. A method according to claim 1 further comprising a step (f) of repeating steps (d) and (e) to provide a dynamic 3D ultrasonic image.

3. A method according to claim 1, wherein the plurality of positions are 3D positions and the positioning system is driven by a motor configured therein.

4. A method according to claim 3, wherein the positioning system transmits respective positioning data of the plurality of 3D positions to a micro processor for an operation.

5. A method according to claim 1, wherein the step (c) further comprises a step (c1) of determining one of the plurality of unit phases being the specific unit phase of step (d).

6. A method according to claim 1 further comprising a step (e1) of displaying the 3D ultrasonic image of the vessel.

7. A method according to claim 1, wherein 20 to 30 ultrasonic images are acquired at each of the plurality of positions in the step (b).

8. A method according to claim 1, wherein the plurality of cardiac cycles are provided by an electrocardiogram simultaneously recorded during the acquiring time periods.

9. A method of imaging a three-dimensional (3D) ultrasonic image, comprising the steps of:
(a) positioning a plurality of positions along a target region;
(b) providing a plurality of ultrasonic images captured at the plurality of positions;
(c) catching a specific ultrasonic image for each of the plurality of positions; and
(d) reconstructing the specific ultrasonic images of the plurality of positions to provide the 3D ultrasonic image.

10. A method according to claim 9, wherein the plurality of ultrasonic images of the plurality of positions are respectively captured during a plurality of capturing time periods each of which is not shorter than a duration of a heartbeat, and the plurality of positions are successive and positioned by an auxiliary system.

11. A method according to claim 10 further comprising a step (b1) of providing a plurality of cardiac cycles respectively corresponding to the capturing time periods, wherein each of the plurality of cardiac cycles is divided into a plurality of unit phases and the specific ultrasonic images correspond to one of the plurality of unit phases.

12. A method according to claim 9 further comprising a step (e) of repeating the steps (c) and (d) to provide a dynamic 3D ultrasonic image.

13. A system imaging a three-dimensional (3D) ultrasonic image of a target region, comprising:
an ultrasonic transducer providing a plurality of ultrasonic images classified into a plurality of image sets each of which has specific ultrasonic images revealing a specific position of the targeting region;
a positioning system positioning the specific positions; and
a processor generating the 3D ultrasonic image by reconstructing from a respective first one of the specific ultrasonic images of each of the plurality of image sets.

14. A system according to claim 12 further comprising an electrocardiograph providing an electrocardiogram having a plurality of cardiac cycles each of which has a plurality of unit phases, wherein the respective first ultrasonic images correspond to a first one of the plurality of unit phases.

15. A system according to claim 12, wherein the processor generates a dynamic 3D ultrasonic image from reconstructing a respective second one of the specific ultrasonic images of each of the plurality of image sets.

16. A system according to claim 12, wherein the target region is one of a vessel and an organ.

17. A system according to claim 12, wherein the positioning system provides respective 3D information of the specific positions and respective 3D information of the plurality of ultrasonic images, and transmits the respective 3D information of the specific positions and the plurality of ultrasonic images to the processor to generate the 3D ultrasonic image.

18. A system according to claim 12, wherein the positioning system has a driving motor and a spring device.

19. A system according to claim 12, wherein the specific positions are successive along the target region.

20. A system according to claim 12, wherein each of the plurality of image sets includes 20 to 30 ultrasonic images.
